(19) United States
(12) Patent Application Publication
(10) Pub. No.: US 2003/0119208 A1
(43) Pub. Date: Jun. 26, 2003

(54) Electrochemical immunosensor and kit and method for detecting biochemical anylyte using the sensor

(76) Inventors: Hyun Chul Yoon, Seoul (KR); Haesik Yang, Daejeon (KR); Chi-Hoon Jun, Daejeon (KR); Youn Tae Kim, Daejeon (KR)

Correspondence Address: BLAKELY SOKOLOFF TAYLOR & ZAFMAN, 12400 WILSHIRE BOULEVARD, SEVENTH FLOOR, LOS ANGELES, CA, 90025, US

(21) Appl. No.: 10/308,435

(22) Filed: Dec. 2, 2002

(30) Foreign Application Priority Data
Dec. 4, 2001 (KR) 2001-76229

(51) Int. Cl. G01N 33/551

(52) U.S. Cl. 436/524

(57) ABSTRACT

An electrochemical immunosensor including a biological sensor layer with an antigen or a ligand residue immobilized thereon, and a biochemical analyte detection kit and method for electrochemically signaling a biological reaction occurring in the biological sensor layer are provided. The electrochemical immunosensor includes a substrate, an electrode or an electrode array formed on the substrate, and a biological sensor layer formed on the electrode or the electrode array and including a polymeric dendrimer monolayer with an antigen or a ligand residue immobilized on the surface thereof. The biological sensor layer further includes an adhesive layer for biomolecular immobilization between the electrode and the polymeric dendrimer monolayer.

### BACKGROUND OF THE INVENTION

This application claims the priority of Korean Patent Application No. 2001-76229, filed Dec. 4, 2002, in the Korean Intellectual Property Office, the disclosure of which is incorporated herein in its entirety by reference.

1. Field of the Invention

The present invention relates to a biosensor and an electrochemical signal detection method using the same, and more particularly, to an electrochemical immunosensor and an array-type electrochemical immunosensor utilizing biological affinity recognition interactions occurring in a biological sensor layer and catalytic precipitation induced by the enzyme-catalyzed reaction and a kit and a method for detecting biochemical analytes using the electrochemical immunosensor and the array-type electrochemical immunosensor.

2. Description of the Related Art

Many efforts have been made to develop biospecific affinity recognition biosensors. Recently, the field of biosensors continues to make starling progress in connection with electronic/information communications engineering fields. Also, there is an increasing demand for the development of micro-electrodes and electrochemical detecting techniques for miniature sensors. The research and development of miniature biospecific affinity-sensing biosensors is in the stage of expansion, and there is an increasing need for the detection of a variety of biochemical species such as proteins or ligands.

The implementation of biospecific affinity-sensing biosensors relies on the efficient immobilization of biochemical species involved in biological reactions, which should be discriminated from test-tube reactions, on a narrow area of a small sensor electrode, and the efficient transduction of the key biological affinity recognition reaction derived at the surface of the sensor electrode. In other words, the target is to develop efficient techniques of immobilizing a particular protein or ligand of interest on a small chip surface area and to develop techniques of detecting diverse biological interactions. Technical requirements for the efficient immobilization to lead effective biological affinity interactions include (1) the numerical optimization of the density of biological probe ligands on the electrode surface, (2) the orientation of biological entities for the maximum efficiency of biological affinity recognition interactions, and (3) the suppression of non-specific adsorption. In addition, the efficient transduction of the biological affinity interactions necessitates highly sensitive and accurate signal detection.

Many studies on the efficient biological entities immobilization have been done, and the recent tendency is towards the self-assembled monolayer based immobilization. More recently, an immobilization method taking advantages of both the self-assembled monolayer and conventional polymer based immobilization methods, which provide a high immobilization yield using polymeric dendrimer, has been published (Yoon et al., Analytical Biochemistry, 282 (2000), 121, Langmuir, 17 (2001), 1234).

However, there is still a need for a more advanced, highly sensitive, accurate, and easy-to-quantify signal detection method for the effective transduction of biological affinity recognition interactions.

Recently, array-type electrochemical immunosensors for high throughput and multiple analytes become interesting. In the electrochemical array-type immunosensor, a biochemical analyte detection method for reducing crosstalk between adjacent electrodes constituting the electrode array is required.

### SUMMARY OF THE INVENTION

Accordingly, the invention provides an electrochemical immunosensor capable of accurately and conveniently detecting signals from biological affinity immune interactions derived in a biological sensor layer.

The invention also provides a biochemical analyte detection kit capable of accurately detecting highly sensitive signals in the electrochemical transduction of biological interactions derived in a biological sensor layer and capable of conveniently quantifying the detected signals.

The invention also provides a biochemical analyte detection method for accurately and conveniently detecting highly sensitive electrochemical signals from biological interactions derived in a biological sensor layer.

The invention also provides a biochemical analyte detection method for minimizing crosstalk between adjacent electrodes constituting the electrode array of an array-type electrochemical immunosensor.

In an aspect, the invention provides an electrochemical immunosensor comprising: a substrate; an electrode or an electrode array formed on the substrate; and a biological sensor layer formed on the electrode or the electrode array and including a polymeric dendrimer monolayer with an antigen or a ligand residue immobilized on the surface thereof.

In the electrochemical immunosensor according to the present invention, the substrate may be silicon or glass, and the electrode may be formed of gold.

When an antigen is immobilized on the surface of the polymeric dendrimer monolayer, the antigen may have a functional group such as succinimide or aldehyde. When a ligand residue is immobilized on the surface of the polymeric dendrimer monolayer, the ligand residue may be biotin.

In the electrochemical immunosensor according to the present invention, the biological sensor layer may further comprise an adhesive layer for biomolecular immobilization between the electrode and the polymeric dendrimer monolayer. In this case, the adhesive layer for biomolecular immobilization may be formed as a self-assembled monolayer basically including thiol or amine group.

In another aspect, the invention provides a biochemical analyte detection kit comprising: an electrochemical immunosensor having a biological sensor layer including a self-assembled monolayer formed on an electrode and a polymeric dendrimer monomer with an antigen or a ligand residue immobilized on the surface thereof; a buffer solution as a dilution of an antibody or a receptor capable of specifically binding to the antigen or the ligand residue in the polymeric dendrimer monolayer, respectively; a precipitation substrate; and a labeled catalytic enzyme capable of binding to an antibody or a receptor to discriminate whether a specific interaction between the antigen and the antibody or between the ligand residue and the receptor has occurred and inducing precipitation from the precipitation substrate.

The antigen is immobilized on the polymeric dendrimer monolayer and an antibody capable of specifically binding to the antigen is diluted in the buffer solution. The ligand residue is immobilized on the polymeric dendrimer monolayer and a receptor capable of binding to the ligand residue is diluted in the buffer solution. For example, when the ligand residue is biotin, the receptor may be avidin or streptavidin.

In the biochemical analyte detection kit according to the present invention, the precipitation substrate may be formed of 4-chloro-1-naphthol. The labeled catalytic enzyme may be peroxidase, alkaline phosphatase, or glucose oxidase.

In another aspect, the invention provides a method for detecting and quantifying a biochemical analyte in a liquid sample using the electrochemical immunosensor and the array-type electrochemical immunosensor described above, the method involving reacting the liquid sample with the biological sensor layer. Next, a mixture solution of a precipitation substrate and a labeled catalytic enzyme is applied to the surface of the biological sensor layer to induce a precipitate formation reaction. An electrochemical signal is detected from the electrode of the electrochemical immunosensor using a cyclic voltammetric method. In detecting the electrochemical signal comprises, a change in the voltage-current waveform or maximum current value obtained from the detected electrochemical signal is read to measure the attenuation of the electrochemical signal due to a reduction in the effective electrode area of the electrochemical immunosensor.

According to the present invention, signals from the biological immunoreaction occurring in the biological sensor layer are detected using the electrochemical immunosensor, wherein precipitation is induced by biospecific affinity recognition reaction, so that the detection of the sensor signal is more accurate, convenient, and sensitive and can be easily quantified. Also, crosstalk between adjacent electrodes constituting the electrode array in the array-type immunosensor can be minimized.

### BRIEF DESCRIPTION OF THE DRAWINGS

The above features and advantages of the present invention will become more apparent by describing in detail exemplary embodiments thereof with reference to the attached drawings in which:

FIG. 1 shows the structure of main parts of an electrochemical immunosensor according to an embodiment of the present invention;

FIG. 2 is a schematic view illustrating the structure of a biological analyte detection kit according to an embodiment of the present invention and the principles of signalling in the biological analyte detection kit;

FIG. 3 shows photographs of the electrode surfaces showing negative and positive responses to the precipitation reaction induced from a precipitation substrate in a biochemical analyte detection kit according to the present invention;

FIG. 4 shows the results of electrochemical signal detection for biochemical analytes after a precipitation reaction from the precipitation substrate in the biochemical analyte detection kit according to the present invention;

FIG. 5 is a graph showing the magnitude of a sensor signal versus the concentration of antibody used, which is the result of electrochemical quantification of biospecific affinity recognition reactions derived at the surface of the electrode according to a biochemical analyte detection method according to the present invention; and

FIG. 6 illustrates the structure of main parts of an array-type electrochemical immunosensor according to another embodiment of the present invention and the principles of signalling in the array-type electrochemical immunosensor.

### DETAILED DESCRIPTION OF THE INVENTION

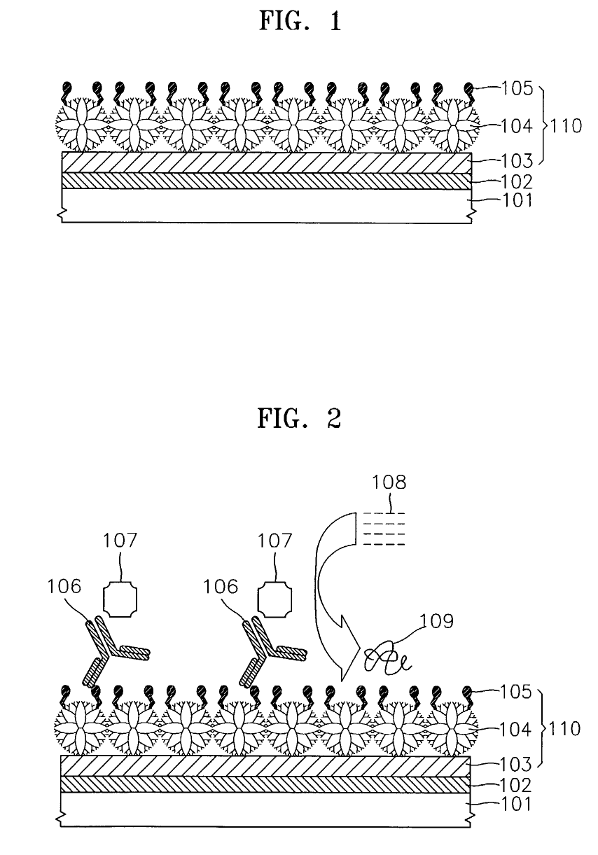

The main structure of an electrochemical immunosensor according to an embodiment of the present invention is shown in FIG. 1. Referring to FIG. 1, the electrochemical immunosensor according to the present invention includes a substrate 101, an electrode 102 formed on the substrate 101, and a biological sensor layer 110. The substrate 101 may be formed of silicon or glass. The electrode 102 may be formed of a thin gold film using an evaporation or sputtering method. When the electrode 102 is formed as a thin gold film, a self-assembled monolayer basically including thiol or amine group can be easily formed on the electrode 102. The biological sensor layer 110 formed on the electrode 102 includes a self-assembled monolayer 103, which acts as an adhesive for biomolecular immobilization, and a polymeric dendrimer monolayer 104 with an antigen 105 or ligand residue immobilized on the surface thereof. The biological sensor layer 110 is formed within a range of several nanometers from the surface of the electrode 101. The self-assembled monolayer 103 includes thiol or amine group in its basic structure. The polymeric dendrimer monolayer 104 may be formed using carbodiimide. In other words, the antigen 105 or ligand residue can be immobilized through general chemical reactions with the amine group in the polymeric dendrimer monolayer 104.

The antigen 105 immobilized on the surface of the polymeric dendrimer monolayer 104 has a functional group, including succinimide or aldehyde. When a ligand residue is immobilized on the surface of the polymeric dendrimer monolayer 104, the ligand residue may be biotin.

FIG. 2 is a schematic view illustrating the structure of a biological analyte detection kit according to an embodiment of the present invention, which includes the electrochemical immunosensor described with reference to FIG. 1, and illustrating biospecific affinity interactions and precipitate formation reaction derived in the surface of the biological sensor layer 110.

Referring to FIG. 2, the biochemical analyte detection apparatus according to the present invention includes an electrochemical immunosensor including the biological sensor layer 110 on the electrode 101, a buffer solution as a dilution of an antibody 106 or receptor capable of specifically binding to the antigen 106 or ligand residue, respectively, immobilized on the polymeric dendrimer monolayer 104 of the biological sensor layer 110, a precipitation substrate 108, and a labeled catalytic enzyme 107 capable of binding to the antibody 106 or the receptor to discriminate whether a specific interaction between the antigen 105 and the antibody 106 or between the ligand residue and the receptor has occurred and inducing precipitate formation reaction from the precipitation substrate 108.

When a ligand residue is immobilized on the polymeric dendrimer monolayer 104, a buffer solution as a dilution of a receptor that can specifically bind to the ligand residue is used. For example, when biotin is immobilized as the ligand residue, avidin or streptavidin may be used as the receptor.

4-chloro-1-naphthol is more useful for the precipitation substrate 108. The labeled catalytic enzyme 107 may be peroxidase, alkaline phosphatase, or glucose oxidase.

The biological sensor layer 110 on which the antigen 105 or ligand residue has been immobilized performs biosensing through biospecific affinity interactions with the antibody 106 or receptor in a biochemical analyte. In FIG. 2, a biospecific binding of the antibody 106 to the immobilized antigen 105 is illustrated. The labeled catalytic enzyme 107 for the detection of whether biospecific interactions have occurred or not, for example, peroxidase, is bound to the antibody 106 and catalyzes the biological interaction for biosensor signal detection.

The antibody protein bound to the labeled catalytic enzyme 107 can be qualified from color changes on the electrode surface, which occur due to the change of the precipitation substrate 108 by the labeled catalytic enzyme 107, for example, peroxidase. Sensor signals from the antibody protein can be quantitatively measured using an electrochemical method.

The biochemical change of the labeled catalytic enzyme 107 generates precipitate 109 on the surface of the electrode 101. A thin film of the precipitate 109 is formed on the surface of the electrode 101, and the surface color of the electrode 101 visibly changes.

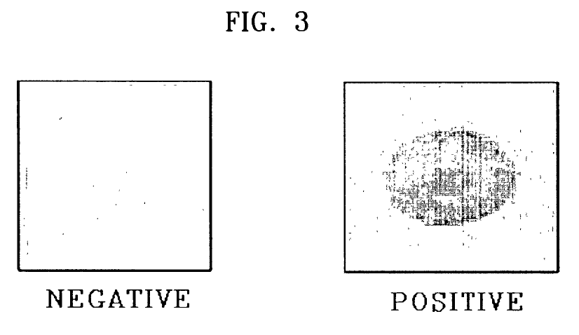

FIG. 3 shows photographs of the electrode surfaces showing negative and positive responses to the precipitation reaction induced from the precipitation substrate 108. In FIG. 3, when a biospecific interaction is induced at a circular center region of the electrode of the electrochemical immune sensor, a thin film of precipitate appears on the surface of the electrode for a positive response sample. However, in a negative response sample, no change is observed from the electrode surface before and after precipitation reaction.

As described above, biochemical analytes in a liquid sample can be detected and quantified as follows using the electrochemical immuno-sensor according to the present invention having the structure described above In particular, a dilute liquid sample containing about several micrograms of the antibody 106 or ligand residue per mililiter is reacted with the biological sensor layer 110 of the electrochemical immunosensor. To this end, the dilute liquid sample containing the antibody 106 or ligand residue is pipetted onto biological sensor layer 110 on the electrode 101 and left for a predetermined period of time, for example, about 10 minutes to allow binding reactions. After the reaction, the surface of the biological sensor layer 110 on the electrode 101 is washed with saline buffer solution. Next, a solution mixture of the precipitation substrate 108 and the labeled catalytic enzyme 107 is dropped onto the surface of the biological sensor layer 110 and stayed for a few minutes to induce precipitation. When the result of the precipitation reaction is positive, a thin film of the precipitate 109 is formed on the surface of the electrode 101.

After the precipitate formation reaction, electrochemical signals are detected from the electrode 101 of the electrochemical immunosensor. Detection of the electrochemical signals will be described in detail below.

A cyclic voltammetric method is applied in detecting electrochemical signals from the electrode 101 of the electrochemical immunosensor. The cyclic voltammetric method is a widely used electrochemical signal detection method that can be achieved with a simple, economical sensor and system, compared with other detection methods, including spectrometry. In detecting electrochemical signals from the electrode 101 using the cyclic voltammetric method, a three-electrode configuration with a working electrode corresponding to the electrode 101 described above, a silver/silver chloride reference electrode, and a platinum wire auxiliary electrode is used for signal detection. In the cyclic voltammetric method, electrically active water-soluble species are useful as signal tracers. Ferrocene derivatives, such as ferrocene methanol, are generally used for the electrochemically active species. A few millimoles of electrochemically active species dispersed in an electrolyte is used. In order to measure the attenuation of an electrical signal due to a reduction in the effective electrode area of the electrochemical immunosensor, changes in the voltage-current waveform or maximum current value before and after the reaction for precipitation, which are obtained using the electrochemical signals detected from the same sensor electrode by the cyclic voltammetric method, are measured and quantified as a numerical value.

As described above, the biochemical analyte detection method according to the present invention is based on the generation and precipitation of insoluble precipitates by the antibody immobilized on the electrode surface through biospecific affinity interactions and the labeled catalytic enzyme bound to the antibody, and the accompanying reduction in the effective electrode area due to the insoluble precipitates.

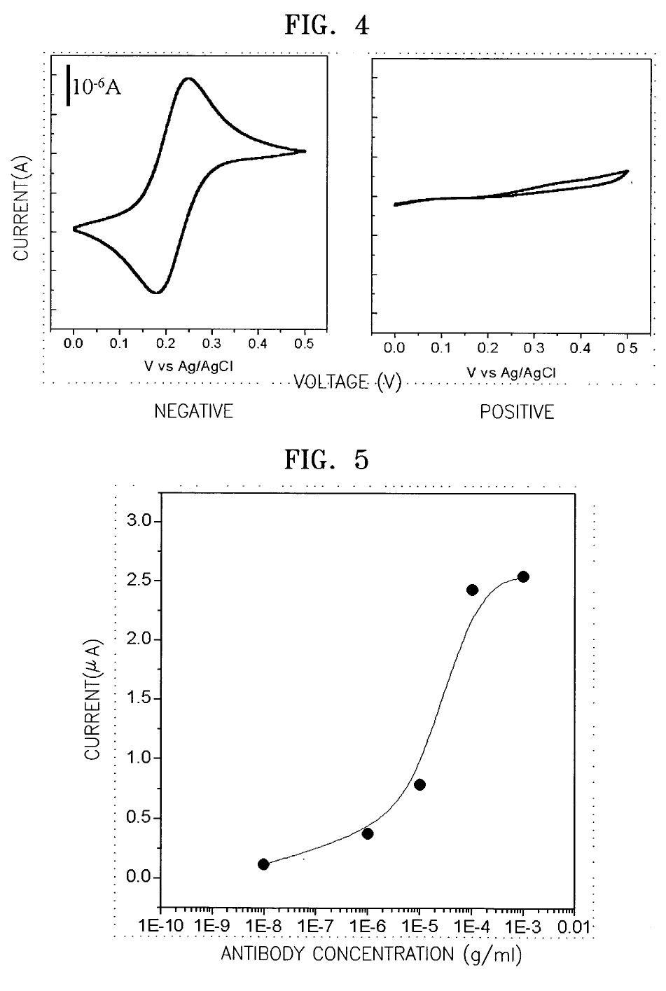

FIG. 4 shows graphs of negative and positive responses in biochemical analytes after the precipitation induction reaction as described above took place, which were detected using the electrochemical cyclic voltammetric method.

As shown in FIG. 4, when the negative response curve is read from the electrochemical immunosensor, it is considered that there has been no antibody 106 immobilized on the surface of the electrode 101. Accordingly, no precipitation reaction takes place to pile the precipitate 109 on the surface of the electrode 101. As a result, a fully developed cyclic voltammogram for the presence of ferrocene in the electrolyte used for cyclic voltammetry appears as shown in the left graph of FIG. 4. When the positive response curve indicating the presence of the immobilized antibody 106 is read, due to the deposition of the precipitate 109 as a thin layer on the surface of the electrode 101, as described above, the electrochemical active species cannot access the surface of the electrolyte 101 resulting in a cyclic voltammogram that is typical of an insulating film, as shown in the right graph of FIG. 4.

FIG. 5 is a graph showing the magnitude of a sensor signal versus the concentration of antibody used, which is used in electrochemical quantification of biospecific affinity interactions derived at the surface of the electrode according to the biochemical analyte detection method according to the present invention. As is apparent from FIG. 5, the biospecific affinity interaction at the electrode surface can be electrochemically quantified using the precipitation induction method applied in the biochemical analyte detection method according to the present invention.

The biochemical analyte detection method based on the electrochemical signal detection according to the present invention is accurate, highly sensitive, and convenient for implementing the sensor systems. In the electrochemical immunosensor, which may be an array-type including electrodes in an array, according to the present invention, the biological sensor layer is formed within a range of a few nanometers from the surface of the electrode, and a soluble substrate becomes insoluble and is precipitated on that biological sensor layer, so that reverse-diffusion of the reaction product and accompanying signal interference between adjacent electrodes of the electrode array can be prevented. This feature is crucial in array-type immunosensors.

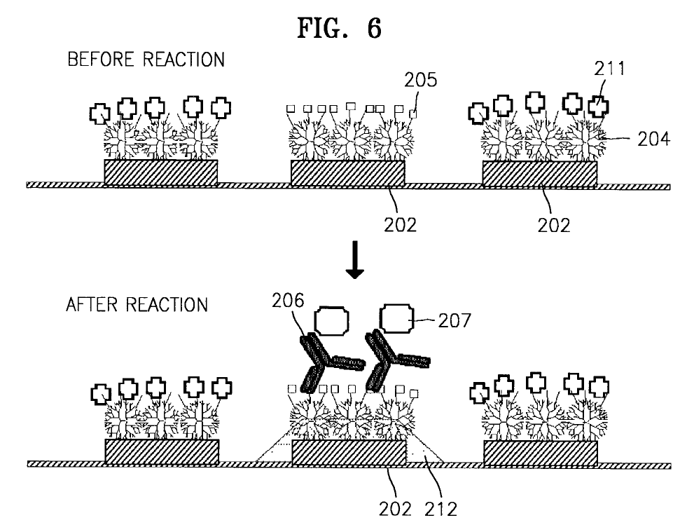

FIG. 6 illustrates the structure of main parts of an array-type electrochemical immunosensor according to another embodiment of the present invention and the principles of signalling in the array-type electrochemical immunosensor.

Referring to FIG. 6, on the surface of each electrode 202 of the electrode array, a first antigen 205 and a second antigen 211 are immobilized to identify multiple analytes. A target antibody 206 is subject to a binding reaction with the first and second antigens 205 and 211 and a precipitation reaction for signal detection. As a result, a deposited film 212 is formed only on the electrode 202 where the biospecific affinity interaction has occurred. In FIG. 6, reference numeral 204 denotes a polymeric dendrimer monolayer, and reference numeral 207 denotes a labeled catalytic enzyme.

As is apparent from the reaction results in FIG. 6, when electrochemical signalling is performed using the array-type immunosensor according to the present invention, crosstalk between adjacent electrodes of the electrode array can be minimized.

The biochemical analyte detection method according to the present invention based on immunoreaction using the labled catalytic enzyme bound to the antibody can be advantageously applied in many clinical fields. Also, an electrochemical method according to the present invention, instead of conventional optical methods, such as absorbency measurement, may be used for signal detection using a simple system at low costs, which is an advantage of the present invention over the conventional methods,

The biochemical analyte detection method according to the present invention is not limited to immunosensors using the reaction with antibody. For example, when biotin, instead of antigen or ligand residue, is immobilized, biospecific affinity interactions of the biotin with avidin or streptavidin can be detected using the method according to the present invention.

According to the present invention, an electrochemical immunosensor and an array-type electrochemical microsensor including a biological sensor layer with a polymeric dendrimer monolayer for the immobilization of an antigen or ligand residue is implemented. Also, an electrochemical signalling method for the biological interaction derived in the biological sensor layer formed on the electrode surface of the electrochemical immunosensor and the array-type electrochemical immunosensor is achieved. In particular, according to the present invention, an immuno-reactive biological sensor layer is formed as a self-assembled monolayer, precipitation is induced at the electrode surface by the catalytic reaction of an immobilized labeled enzyme, and the attenuation of an electrical signal due to a reduction in the effective electrode area resulting from the deposition of the precipitate is electrochemically measured and quantified. According to the present invention, signals from the biological immunoreaction occurring in the biological sensor layer are detected using the electrochemical immunosensor, wherein precipitation is induced by biospecific affinity recognition reaction, so that the detection of the sensor signal is more accurate, convenient, and sensitive and can be easily quantified.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An electrochemical immunosensor comprising:
a substrate;
an electrode or an electrode array formed on the substrate; and
a biological sensor layer formed on the electrode or the electrode array and including a polymeric dendrimer monolayer with an antigen or a ligand residue immobilized on the surface thereof.

2. The electrochemical immunosensor of claim 1, wherein the substrate is silicon or glass.

3. The electrochemical immunosensor of claim 1, wherein the electrode or the electrode array is formed of gold.

4. The electrochemical immunosensor of claim 1, wherein the antigen is immobilized on the surface of the polymeric dendrimer monolayer.

5. The electrochemical immunosensor of claim 4, wherein the antigen contains succinimide or aldehyde functional group.

6. The electrochemical immunosensor of claim 1, wherein the ligand residue is immobilized on the surface of the polymeric dendrimer monolayer.

7. The electrochemical immunosensor of claim 6, wherein the ligand residue is biotin.

8. The electrochemical immunosensor of claim 1, wherein the biological sensor layer further comprises an adhesive layer for biomolecular immobilization between the electrode and the polymeric dendrimer monolayer.

9. The electrochemical immunosensor of claim 8, wherein the adhesive layer for biomolecular immobilization is formed as a self-assembled monolayer basically including thiol or amine group.

10. A biochemical analyte detection kit comprising:
an electrochemical immunosensor having a biological sensor layer including a self-assembled monolayer formed on an electrode and a polymeric dendrimer monomer with an antigen or a ligand residue immobilized on the surface thereof;
a buffer solution as a dilution of an antibody or a receptor capable of specifically binding to the antigen or the ligand residue in the polymeric dendrimer monolayer, respectively;
a precipitation substrate; and
a labeled catalytic enzyme capable of binding to an antibody or a receptor to discriminate whether a specific interaction between the antigen and the antibody or between the ligand residue and the receptor has occurred and inducing precipitation from the precipitation substrate.

11. The biochemical analyte detection kit of claim 10, wherein the self-assembled monolayer basically includes thiol or amine group.

12. The biochemical analyte detection kit of claim 10, wherein the antigen is immobilized on the polymeric dendrimer monolayer, and an antibody capable of specifically binding to the antigen is diluted in the buffer solution.

13. The biochemical analyte detection kit of claim 10, wherein the ligand residue is immobilized on the polymeric dendrimer monolayer, and a receptor capable of specifically binding to the ligand residue is diluted in the buffer solution.

14. The biochemical analyte detection kit of claim 13, wherein the ligand residue is biotin, and the receptor is avidin or streptavidin.

15. The biochemical analyte detection kit of claim 10, wherein the precipitation substrate is formed of 4-chloro-1-naphthol.

16. The biochemical analyte detection kit of claim 10, wherein the labeled catalytic enzyme is peroxidase, alkaline phosphatase, or glucose oxidase.

17. A method for detecting and quantifying a biochemical analyte in a liquid sample using the electrochemical immunosensor of claim 1, the method comprising:
reacting the liquid sample with the biological sensor layer;
applying a mixture solution of a precipitation substrate and a labeled catalytic enzyme to the surface of the biological sensor layer to induce a precipitate formation reaction; and
detecting an electrochemical signal from the electrode of the electrochemical immunosensor using a cyclic voltammetric method.

18. The method of claim 17, wherein the precipitation substrate is formed of 4-chloro-1-naphthol.

19. The method of claim 17, wherein the labeled catalytic enzyme is peroxidase, alkaline phosphatase, or glucose oxidase.

20. The method of claim 17, wherein detecting the electrochemical signal comprises measuring a change in the voltage-current waveform or maximum current value obtained from the detected electrochemical signal to measure the attenuation of the electrochemical signal due to a reduction in the effective electrode area of the electrochemical immunosensor.
